(19) United States
(12) Patent Application Publication
(10) Pub. No.: US 2022/0396591 A1
(43) Pub. Date: Dec. 15, 2022

(54) METHOD FOR PREPARING ALKENYL AMINOBORANES AND THEIR DERIVATIVES, AND USES THEREOF

(71) Applicants: UNIVERSITE DE BORDEAUX, Bordeaux (FR); INSTITUT POLYTECHNIQUE DE BORDEAUX, Talence Cedex (FR); CENTRE NATIONAL DE LA RECHERCHE SCIENTIFIQUE, Paris Cedex 16 (FR)

(72) Inventors: Mathieu PUCHEAULT, Camblanes et Meynac (FR); Virginie LIAUTARD, Begles (FR)

(21) Appl. No.: 17/765,986

(22) PCT Filed: Oct. 2, 2020
(86) PCT No.: PCT/EP2020/077718

§ 371 Date: Apr. 1, 2022

(30) Foreign Application Priority Data
Oct. 4, 2019 (FR) FR1911003

(51) Int. Cl. C07F 7/08; C07F 5/02; B01J 21/06

(52) U.S. Cl. CPC C07F 7/081; C07F 5/025; B01J 21/066

(57) ABSTRACT

Disclosed is a method for preparing alkenyl aminoboranes and their derivatives, and uses thereof. The method for preparing alkenyl aminoboranes includes contacting, preferably in a single synthesis step, a terminal alkyne, an aminoborane and a catalyst chosen from Schwartz's reagent (Cp2ZrHCl), dicyclohexylborane (HBCy), diisopinocamphenylborane (HBipc2) and 9-Borabicyclo(3.3.1)nonane (9-BBN).

### BACKGROUND OF THE INVENTION

### Field of the Invention

The present invention relates to a method for the preparation of alkenylaminoboranes and their derivatives, and their uses.

### Description of the Related Art

Alkenylaminoboranes are compounds exhibiting both the particularities of alkenylboranes and aminoboranes.

Alkenylboranes are versatile synthetic intermediates that find application in many organic synthetic strategies.

They are particularly of great interest as synthesis intermediates, particularly in post-functionalization steps (halogenation, cross-coupling) allowing the introduction of useful chemical functions for obtaining natural or biological products.

Vinyl units are indeed very present in many molecules of biological interest.

The stereoselective synthesis of these compounds is in particular very important because it conditions the properties of the molecules.

1-Alkenylboronates can be obtained by the hydroboration of terminal alkynes.

The hydroboration reaction of terminal alkynes consists of the addition of a boron-hydrogen bond on the triple bond giving rise to the corresponding alkenylborane.

The hydroboration of alkynes can be done without a catalyst but requires the use of specific boranes such as dialkoxyboranes, catecholborane or pinalcolborane.

However, these reagents are expensive and/or unstable.

Catecholborane, for example, is highly unstable to air and humidity and requires purification steps before use. Moreover, the reaction with catecholborane leads to by-products and the relative instability of the catechol alkenylboronates obtained requires further transformation into more stable boron esters. Also, the stereoselectivity of uncatalyzed hydroboration is not general, a mixture of regioisomers can be obtained.

The hydroboration of alkynes can be catalyzed by transition metals, or it can be carried out by heterogeneous catalysis or organocatalysis.

The hydroboration of alkynes catalyzed by transition metals has mainly been studied with pinacolborane and catecholborane, expensive reagents. The hydroboration reaction is stereospecific and the formation of (E)-isomers is favored. A method for obtaining alkenylboranes catalyzed by a transition metal is described in the document (WO2006/132896), but it implements the addition of a dialkoxyborane and does not make it possible to obtain alkenylaminoborane.

Furthermore, these methods for preparing alkenylboranes by hydroboration of alkynes are not entirely satisfactory.

In particular, there is a need to have a method for preparing alkenylaminoboranes, making it possible avoiding the use of expensive reagents required by the methods of the prior art, not requiring the use of unstable compounds and allowing the stereoselective preparation of an alkenylaminoborane or one of its derivatives in a stable state, with high yield and/or excellent purity.

A known method for obtaining aminoboranes is that described in patent EP 1 458 729.

The method described in this patent comprises the reaction between diisopropylaminoborane (DIPOB) of formula (iPr)₂NBH₂ and a compound of formula A-X, in which A may be a vinyl group and X is a halogenated leaving group, in the presence of a palladium catalyst.

The method described in this document implements a substitution of the group X and is not carried out by hydroboration of the triple bond of an alkyne, the vinyl function carried by the group A not being reactive during the method described.

### SUMMARY OF THE INVENTION

One aspect of the present invention is a method for the preparation of alkenylaminoboranes by hydroboration of a terminal alkyne in the presence of an aminoborane and a specific catalyst.

Another aspect of the present invention is the use of an alkenylaminoborane as an intermediate compound for the preparation of various families of boron derivatives, such as alkenyldiaminoboranes, alkenyldialkoxyboranes or alkenylfluoroborates.

Another aspect of the present invention is the use of alkenylaminoboranes and derived families obtained from alkenylaminoboranes, as reaction intermediates for coupling or multistep syntheses.

Another aspect of the present invention is the use of an alkenylaminoborane as an intermediate compound for the stereoselective preparation of bromoalkenes.

The inventors have shown that it is possible to prepare alkenylaminoboranes by bringing a terminal alkyne and an aminoborane into contact in the presence of a specific catalyst.

Thus, the subject of the invention is a method for the preparation of an alkenylaminoborane of the following formula (I):

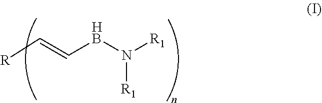

in which R is:
a linear or branched alkyl group of 1 to 18 carbon atoms, optionally bearing at least one substituent,
a linear or branched alkenyl or alkynyl group of 2 to 18 carbon atoms, optionally bearing at least one substituent,
a cycloalkyl or cycloalkenyl group of 3 to 18 carbon atoms, optionally bearing at least one substituent,
a heterocycloalkyl or heterocycloalkenyl group, optionally bearing at least one substituent
an aryl group of 2 to 12 carbon atoms, where the aryl is chosen from the group of aromatics or heteroaromatics, optionally bearing at least one substituent,
an alkyl aryl group, where the aryl is chosen from the group of aromatics or heteroaromatics, optionally bearing at least one substituent,
a halogen chosen from F, Cl, Br, and I,
a silyl group —SiRₐR_{b}R_{c}, —RₐSiR_{b}R_{c}R_{d}, —RₐOSiR_{b}R_{c}R_{d},
—ORₐ, —NHRₐ, —NRₐR_{b}, —SRₐ, —CF₃, —NO₂, —RₐOR_{b}, —RₐNHR_{b}, —RₐNR_{b}R_{c}, —RₐSR_{b} group in which Rₐ, R_{b}, R_{c} and R_{d}, identical or different, represent H, Cl, linear or branched alkyl, alkenyl, alkynyl, cycloalkyl, cycloalkenyl, aryl groups, in particular phenyl, or heterocyclic aromatic or non-aromatic groups, of 1 to 18 carbon atoms, optionally bearing at least one substituent, where said substituents are chosen from:
a linear, branched or cyclic alkyl groups of 1 to 18 carbon atoms,
the halogens F, Cl, Br and I,
OH,

R₁ is a group chosen from isopropyl, cyclohexyl, n is an integer from 1 to 3, comprising bringing into contact, preferably in a single synthesis step: a terminal alkyne, of the following formula:

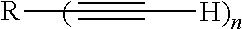

R having the meanings indicated above, of an aminoborane of formula BH₂—N(R₁)₂, and of a catalyst chosen from: Schwartz's reagent (Cp₂ZrHCl), dicyclohexylborane (HBCy), diisopinocamphenylborane (HBipc2), 9-borabicyclo(3.3.1)nonane (9-BBN).

In formula (I), it is understood that when n varies from 1 to 3, the group R has a valence n of alkenylaminoborane functions and the structural possibilities of the group of R are adapted accordingly.

For example when R is a C1 alkyl group, i.e. R has a carbon atom: R is a —CH₃ group if n is equal to 1, R is a —CH₂— group if n is equal to 2 and R is a —CH— group if n is equal to 3.

When R is a C2 alkenyl group, i.e. R has 2 carbon atoms: R is a CH₂═CH group if n is equal to 1, R is a —CH═CH— group if n is equal to 2 and R is a —C═CH— group if n is equal to 3.

When R is a C2 alkynyl group, R is a CH═C— group if n is equal to 1, R is a CH═C group if is equal to 2 and the valence n cannot be equal to 3, because the R group does not cannot carry three alkenylaminoborane functions.

For the amine groups in the variants of R, the alkenylaminoborane function can:
be linked directly to the N atom, for example in the case of —NHRₐ and —NRₐR_{b} groups, -or be linked via Rₐ groups, for example in the case of —RₐNHR_{b} and —RₐNR_{b}R_{c} groups; where we understand that Rₐ cannot be H or Cl.

The same is true for the variant groups of R comprising Si, O or S.

In one embodiment, the invention relates to a method for the preparation of an alkenylaminoborane of formula (I) in which n is equal to 1,

R is a silyl group —SiRₐR_{b}R_{c}, in particular Rₐ, R_{b} and R_{c}, identical or different, are chosen from H, Cl atoms, alkyl groups with 1 to 18 carbon atoms or phenyl groups,

R₁ and R₂ are identical, and have the following formula (I-1-1):

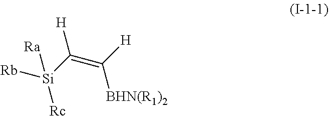

in which R₁ has the meanings indicated above.

The aminoborane of formula BH₂—N(R₁)₂ is preferably chosen from diisopropylaminoborane (DIPOB) and dicyclohexylaminoborane (DICOB).

The steric hindrance provided by the two isopropyl or cyclohexyl substituents prevents, by their arrangement and their volume, the approach of a reagent to the amine function.

In other words, DIPOB and DICOB have a steric hindrance limiting access to the nitrogen atom, thus making the nitrogen atom unreactive.

These two aminoboranes DIPOB and DICOB are notably inexpensive.

The specific catalysts used for the hydroboration of the alkyne by an aminoborane are chosen from among Schwartz's reagent (Cp₂ZrHCl), dicyclohexylborane (HBCy), diisopinocamphenylborane (HBipc2) and 9-borabicyclo(3.3.1)nonane (9-BBN).

These molecules have the following structures:

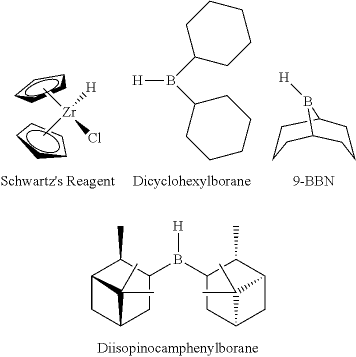

The method for forming the alkenylaminoboranes of formula (I) according to the present invention can be carried out in a single synthesis step, ie a so-called one-pot method.

The use of low-cost raw materials (alkyne, aminoboranes or amine-borane complexes) is particularly advantageous for carrying out the reaction on an industrial scale.

The method for forming the alkenylaminoboranes of formula (I) according to the present invention is stereospecific and forms (E) isomers.

In one embodiment, the method is carried out in the absence of an additive to promote the stereoselectivity of the reaction.

The method according to the invention advantageously makes it possible avoiding the use in the medium of an additive such as an amine to promote stereoselectivity.

In the present invention, the catalyst alone is capable of reacting alkyne and borane to form only (E) isomers.

In one embodiment, the method is performed in the absence of a base.

The method according to the invention advantageously makes it possible avoiding the use in the medium of an additional base which can promote the formation of uncontrolled secondary products.

In the present invention, the catalyst alone is capable of reacting alkyne and borane.

The method according to the invention does not require the addition of a base such as triethylamine (Et₃N) unlike reactions using a transition metal complex as catalyst.

In another embodiment of the method of the invention, n is equal to 1, 2 or 3, preferably n is equal to 1.

In a particular embodiment, the invention relates to a method for the preparation of an alkenylaminoborane of formula (I), in which the method is carried out at a temperature ranging from 20° C. to 80° C., preferably at 70° C.

When the reaction temperature is higher than room temperature, the reaction is carried out with a lower quantity of catalyst than that used for a reaction carried out at room temperature.

Ambient temperature means temperatures of 10° C. to 40° C., in particular of the order of 20° C. to 30° C.

When the reaction temperature is above 80° C., the inventors have observed the presence of impurities such as boron-free alkenes resulting from photodeborylation.

In one embodiment, the invention relates to a method for the preparation of an alkenylaminoborane of formula (I), in which the catalyst is the Schwartz's reagent of formula (C₅H₅)₂ZrHCl or Cp₂ZrHCl.

In one embodiment, the invention relates to a method for the preparation of an alkenylaminoborane of formula (I), in which the catalyst is used in an amount ranging from 0.5% to 20%, in particular from 1% to 12%, preferably 12%.

The amounts are expressed in molar percentage with respect to the limiting reactant.

Advantageously, in one embodiment, the method is carried out in less than 24 hours, preferably in less than one hour.

According to an advantageous embodiment, the method according to the invention is carried out in a solvent, in particular an aprotic solvent, preferably chosen from N,N-Dimethylformamide (DMF), tetrahydrofuran (THF), methylterbutylether (MTBE), diethyl ether (Et₂O), benzene, toluene, xylene, dioxane, or mixtures thereof, in particular MTBE or THF. The method according to the invention allows the use of a wide range of solvents.

Indeed, it can be implemented with solvents usually used in industry.

The solvent can thus be chosen for reasons of cost, toxicity or adaptation to any other synthesis steps.

Advantageously, MTBE and THF with a respective boiling temperature of 66° C. and 55° C., used as solvent in the method of the invention, make it possible to isolate alkenylaminoboranes of formula (I) having a boiling temperature higher than 70° C. by proceeding by evaporation of the solvent, for example using a rotary evaporator.

Advantageously, the invention relates to a method in which the conversion rate of alkyne to alkenylaminoborane is greater than 80%, preferably greater than 97%.

In particular, the yield of the method for preparing the alkenylaminoborane of formula (I) according to the invention is quantitative.

The term “conversion rate” means the rate of terminal alkyne having reacted during the method.

This rate can be determined by analyzing the final product obtained by ¹H NMR.

The comparison of the signal of the propargyl proton, on which the hydroboration reaction is carried out, with that of the other protons of the alkyne serving as a reference, makes it possible to evaluate the quantity of alkyne having reacted during the method according to the invention.

In a variant, the alkenylaminoborane of formula (I) obtained according to the method of the invention does not require an additional purification step because the purity of the product obtained is greater than 90%, in particular greater than 91%, 92%, 93%, 94%, 95%, 96%, 97%, 98% or 99%.

Advantageously, the alkenylaminoborane is obtained with a purity allowing other reactions to be carried out directly in the same reaction medium in order to obtain other families of boron derivatives.

The alkenylaminoborane of formula (I) can be isolated by simple filtration of the reaction medium on kielselghur or on diatomaceous earth, preferably on Celite®, using an eluent.

within the meaning of the present invention the term “purification step” means any step following the synthesis step making it possible to increase the purity of the product.

Examples of purification steps include liquid chromatography, high performance liquid chromatography, recrystallization or distillation.

The purification steps do not include the step of filtration of the mixture, for example on kielselghur or on diatomaceous earth, and evaporation of the solvent.

Advantageously, the alkenylaminoborane of formula (I) can be isolated in liquid or solid form with a purity of the product greater than 90%, in particular greater than 91%, 92%, 93%, 94%, 95%, 96%, 97%, 98% or 99%.

The aminoborane used in the method of the invention can be obtained commercially or by synthesis.

It can also be generated from an amine-borane complex during the hydroboration reaction.

In another embodiment, the invention relates to a method for the preparation of an alkenylaminoborane of formula (I) in which the aminoborane of formula BH₂—N(R₁)₂ is formed in situ by a dehydrogenation reaction of a complex amine-borane of formula H₃B←NH(R₁)₂, preferably in a single synthesis step. For example, the amine-borane dehydrogenation reaction H₃B←NH(R₁)₂ is carried out using an organomagnesium.

Within the meaning of the present invention, the term “amine-borane complex” of formula H₃B←NH(R₁)₂ means a compound comprising a BH₃ group whose vacant p orbital is filled by the pair of electrons of an amine NH(R₁)₂.

Mention may be made, by way of example of an amine-borane complex, of diisopropylamine-borane (DIPAB) of formula H₃B←NH(iPr)₂ or dicyclohexylamine-borane (DICAB) of formula H₃B←NH(Cy)₂.

Within the meaning of the present invention, the term “formed in situ” means the fact that the aminoborane is formed directly during the implementation of the method by mixing the amine-borane complex and an organomagnesium, for example, during the reaction of hydroborylation. The method of the invention can thus be carried out in a single simultaneous step of formation of the aminoborane and hydroborylation of the alkyne.

In an advantageous mode of the method of the invention, an organomagnesium is used for the in situ generation of the aminoborane from the amine-borane complex and is a Grignard reagent, preferably PhMgBr or CH₃MgBr.

Amine-borane complexes are known for their stability towards water, air and light.

They are relatively simple to produce and can be stored for a long time.

It is thus possible to select amine-borane complexes, some of which are more chemically stable and/or commercially available than their aminoborane homologs.

Advantageously, in another embodiment, the subject of the method is the preparation of an alkenylaminoborane of formula (I), in which formula (I) corresponds to one of the following structures:

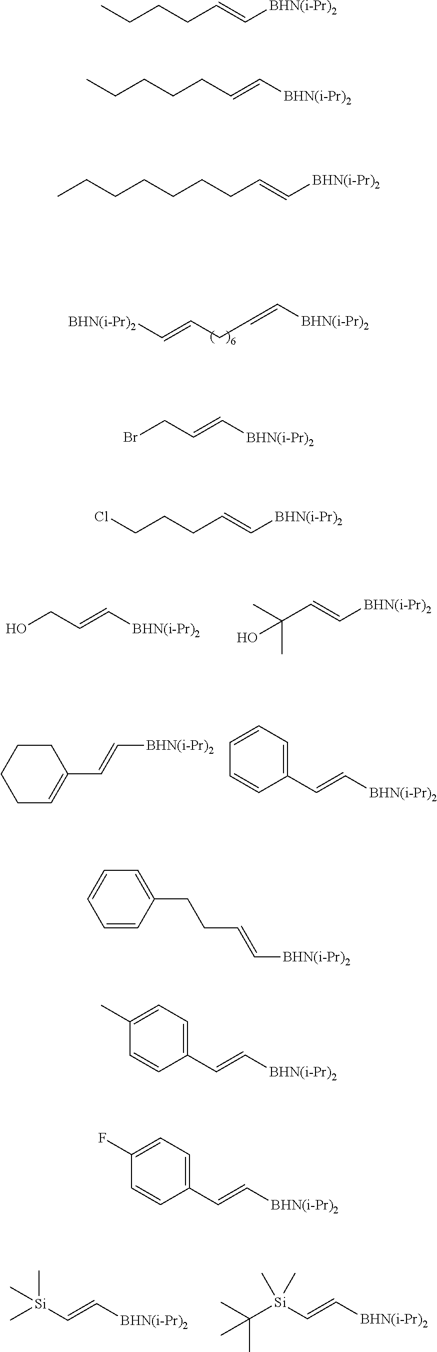

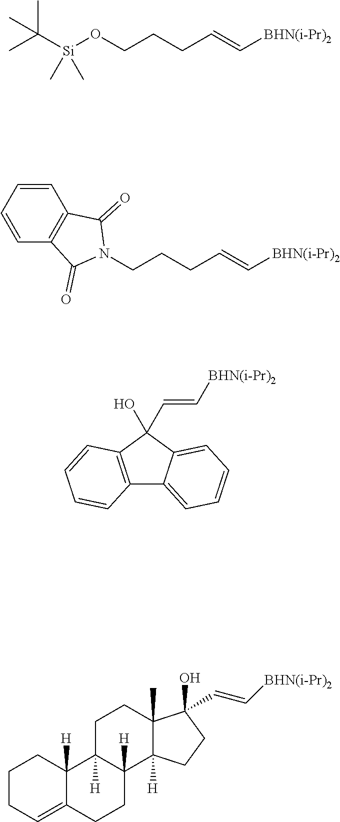

Another subject of the invention is the use of a terminal alkyne, diisopropylaminoborane (DIPOB) or dicyclohexylaminoborane (DICOB) and a catalyst chosen from among Schwartz's reagent (Cp2ZrHCl), dicyclohexylborane (HBCy), diisopinocamphenylborane (HBipc2), 9-borabicyclo(3.3.1)nonane (9-BBN), for carrying out a method for the preparation of an alkenylaminoborane of formula (I) according to the invention.

Another object of the invention is the use of diisopropylaminoborane (DIPOB) or dicyclohexylaminoborane (DICOB) for the implementation of a method for the preparation of an alkenylaminoborane of formula (I) according to the invention.

Another object of the invention is the use of a catalyst chosen from Schwartz's reagent (Cp₂ZrHCl), dicyclohexylborane (HBCy), diisopinocamphenylborane (HBipc2), 9-borabicyclo(3.3.1)nonane (9-BBN), for the implementation of a method for the preparation of an alkenylaminoborane of formula (I) according to the invention.

Another object of the invention is the use of Schwartz's reagent (Cp₂ZrHCl), for the implementation of a method for the preparation of an alkenylaminoborane of formula (I) according to the invention.

Without being bound by theory, a reaction mechanism is proposed according to the scheme below for a method for the preparation of an alkenylaminoborane of formula (I) implementing DIPOB and the Schwartz's reagent.

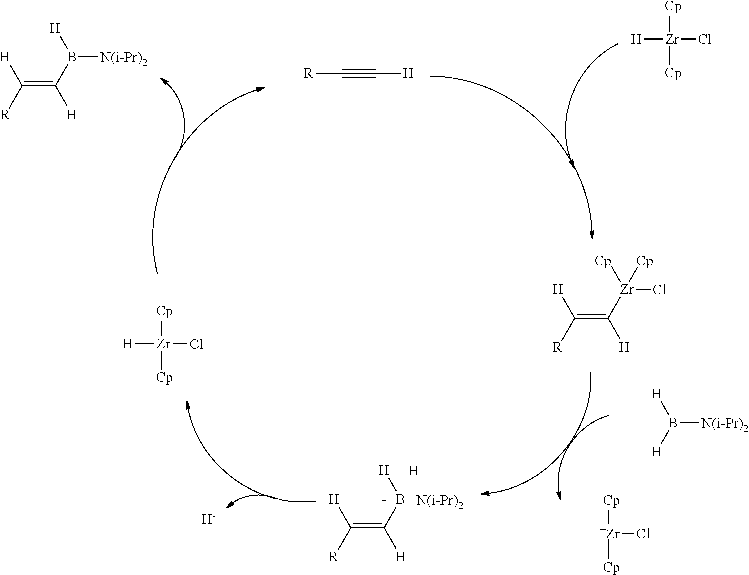

During the method, a step of hydrozirconation of the alkyne by the Schwartz's reagent (syn-addition) takes place.

It is followed by the addition of DIPOB. The addition is done with retention of configuration.

The zirconium diisopropylaminoborohydride thus formed can then release a hydride into the medium to form the alkenylaminoborane of formula (I).

The hydride is immediately captured by the positively charged zirconium derivative.

The Schwartz's reagent is thus regenerated and the catalytic cycle is initiated.

The invention also relates to the use of an alkenylaminoborane of formula (I) prepared according to the method of the invention for the preparation of one of the compounds of formulas (II), (III) or (IV) below:

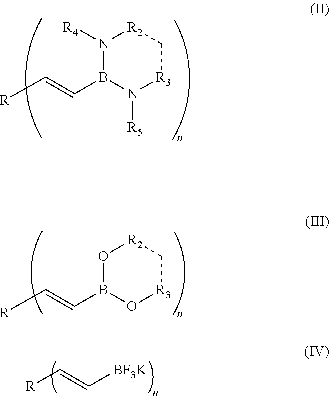

in which R and n have the meanings indicated above, R₂, R₃, R₄, and R₅, are identical or different and represent hydrogens, linear or branched alkyl, alkenyl, alkynyl, cycloalkyl, cycloalkenyl, aryl groups, or aromatic or non-aromatic heterocyclic groups, with 1 to 18 carbon atoms, optionally bearing at least one substituent, where said substituents are chosen from linear, branched or cyclic alkyl groups with 1 to 18 carbon atoms, and halogens F, Cl, Br and I, where R₂ and R₃ can be connected to form a ring together.

Preferably by implementing a one-pot method.

Another object of the present invention relates to a method for the preparation of an alkenyldiaminoborane compound of formula (II):

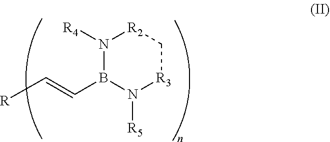

in which

R and n have the meanings indicated above,

R₂, R₃, R₄, and R₅ have the meanings given above, including the following steps:

a) a step of preparing an alkenylaminoborane of formula (I) according to the invention,

b) a step of alcoholysis with an R′—OH alcohol of the alkenylaminoborane of formula (I) into an intermediate alkenylboronate of formula (I-M):

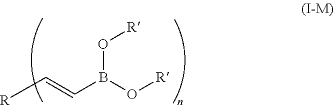

in particular the alcohol R′—OH is chosen from MeOH, EtOH, PrOH or iPrOH, c) a step of substituting the alkoxyl groups R′O of the compound of formula (I-M) with an amino group comprising the groups R₂, R₃, R₄ and R₅ where R₂, R₃, R₄, et R₅ have the meanings indicated above, making it possible to obtain the compound of formula (II), d) optionally a purification step.

Preferably steps a), b) and c) are carried out in one-pot.

Preferably, the alcohol used in step b) alcoholysis is methanol.

The alcoholysis is advantageously carried out using 3 equivalents of alcohol molecule per alkenylaminoborane function.

For example, methanolysis is carried out at low temperature (−40° C.) by adding 3 equivalents of a molecule of methanol per alkenylaminoborane function to the reaction medium.

In another embodiment, the amino group is a diamine containing the groups R₂, R₃, R₄ and R₅.

The amino group is advantageously bidentate and the R₂ and R₃ groups are linked together to form a cycle.

In another embodiment, the invention relates to a method for the preparation of alkenyldiaminoborane compounds of formula (II), in which said amino group is diaminonaphthalene of the following formula:

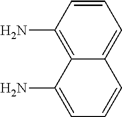 [SMILES: Nc1cccc2cccc(N)c12]

By way of example, step c) of substituting the alkoxyl groups with diaminonaphthalene (dan) is carried out in the presence of FeCl₃ and imidazole in a mixture of solvents MeCN:H₂O (1:1) at room temperature (RT) for 4 hours.

Another object of the present invention relates to a method for the preparation of an alkenylboronate compound of formula (III):

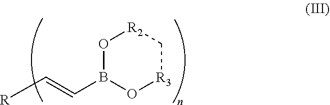

in which

R and n have the meanings indicated above,

R₂ and R₃ have the meanings indicated above, comprising the following steps:

a) a step of preparing an alkenylaminoborane of formula (I) according to the invention;

b) a step of alcoholysis by an alcohol R′—OH of the alkenylaminoborane of formula (I) into an intermediate alkenylboronate of formula (I-M):

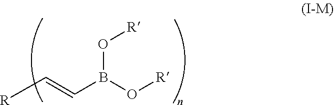

in particular the alcohol is chosen from MeOH, EtOH, PrOH or iPrOH;

c) a step of substituting the alkoxyl groups R′O of the compound of formula (I-M) with an alcohol or diol comprising R₂ and R₃ groups, in particular by a transesterification reaction;

d) optionally a purification step; preferably steps a), b) and c) are carried out in one-pot.

Advantageously, the alcohol used in step b) alcoholysis is methanol.

The alcoholysis is advantageously carried out using 3 equivalents of alcohol molecule per alkenylaminoborane function.

In another embodiment, a diol containing the R₂, R₃ groups is used during step c) of substitution.

The diol group is advantageously bidentate and the R₂ and R₃ groups are linked together to form a cycle.

In a particular embodiment of the invention, the substitution of the alkoxyl groups is carried out by a transesterification reaction.

For example, transesterification is carried out by adding alcohol or diol in diethyl ether at −40° C. to the reaction medium and the solution is brought from −40° C. to ambient temperature.

In a particular embodiment, the invention relates to a method for the preparation of an alkenylboronate compound of formula (III), in which said diol used is pinacol or neopentylglycol.

Advantageously, the method for preparing an alkenylboronate compound of formula (III), in which said diol used is pinacol or neopentylglycol does not require a step for purifying the final product.

A simple drying step by adding a drying agent, for example Na₂SO₄, followed by filtration and concentration by evaporation of the solvent, makes it possible to obtain pure products.

Another object of the invention relates to a method for the preparation of an alkenylfluoroborate compound of formula (IV):

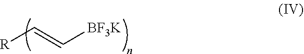

in which

R and n have the meanings indicated above, comprising the following steps:

a) a step of preparing an alkenylaminoborane of formula (I) according to the invention,

b) a step of alcoholysis by an alcohol R′—OH of the alkenylaminoborane of formula (I) into an intermediate alkenylboronate of formula (I-M):

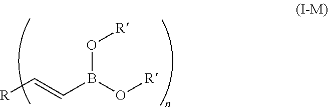

in particular the alcohol R′—OH is chosen from MeOH, EtOH, PrOH or iPrOH;

c) a step of substituting the alkoxyl groups R′O of the compound of formula (I-M) with a fluorinated group, in particular carried out by bringing into contact with KHF₂;

d) optionally a purification step; preferably steps a), b) and c) are carried out in one-pot.

Advantageously, the alcohol used in step b) alcoholysis is methanol.

The alcoholysis is advantageously carried out using 3 equivalents of alcohol molecule per alkenylaminoborane function.

By way of example, step c) of transformation of the intermediate alkenylboronate of formula (IM) into potassium trifluoroborate salt by substitution of the alkoxyl groups, is carried out by adding to the reaction medium a solution of KHF₂ prepared in methanol at −40° C. and the mixture is brought from −40° C. to room temperature (RT).

Advantageously, in one embodiment, the subject of the method is the preparation of one of the compounds of formulas (II), (III) or (IV), corresponding to one of the following structures:

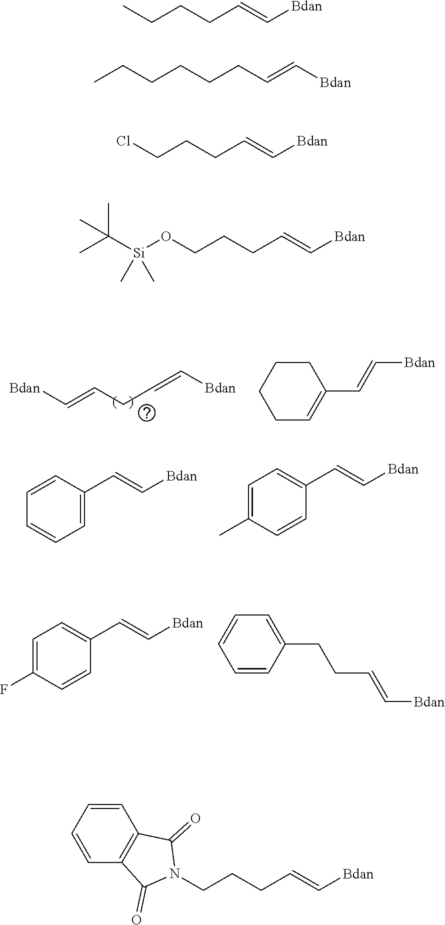

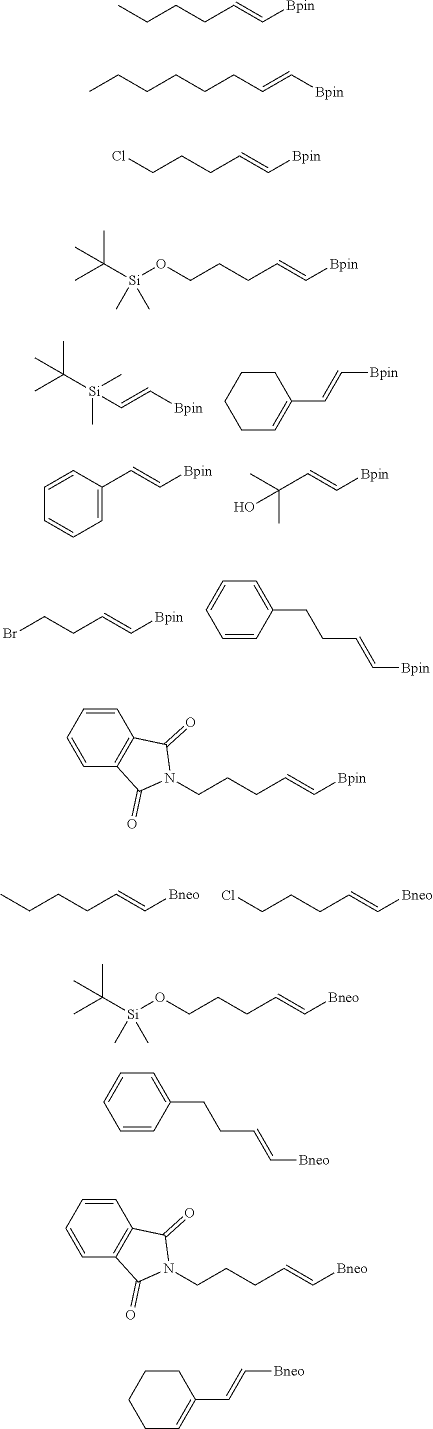

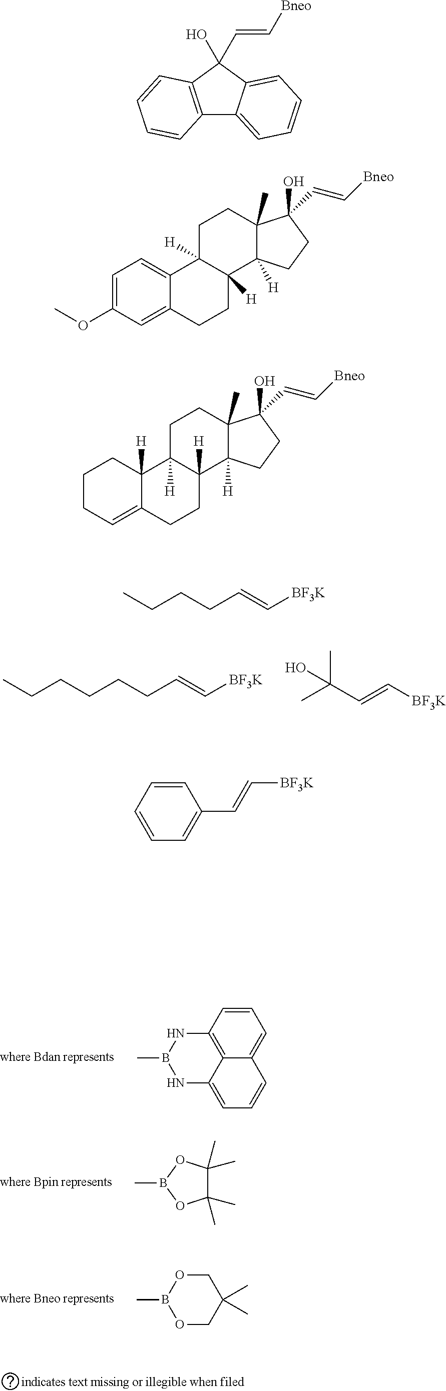

Another object of the invention is the use of the compounds of formulas (I), (II), (III) or (IV) prepared according to one of the methods of the invention, as reaction intermediate compounds, in particular for the implementation of stereoselective, multistep or coupling syntheses, in particular for Suzuki-Miyaura, Chan-Lam, Petasis and halogenation reactions.

Another object of the invention is the use of the method according to the invention for the implementation of the preparation of the compounds of formulas (I), (II), (III) or (IV), as intermediate reaction compounds, in particular for the implementation of stereoselective, multistep or coupling syntheses, in particular for Suzuki-Miyaura, Chan-Lam, Petasis and halogenation reactions.

The invention also relates to the use of an alkenylaminoborane of formula (I) for carrying out a stereoselective synthesis of bromoalkenes (Z) or (E).

In another embodiment, the invention also relates to the use of an alkenylaminoborane of formula (I) prepared according to the method of the invention for the implementation of a stereoselective synthesis of bromoalkenes (Z) or (E).

Another object of the present invention relates to a method for the stereoselective preparation of bromoalkenes (Z) or (E) of the following formulas:

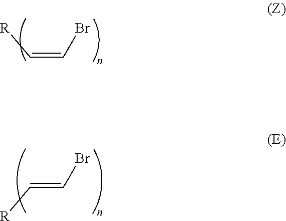

in which R and n have the meanings indicated above, comprising the following steps:

a) a step d alcoholysis by an alcohol R′—OH of an alkenylaminoborane of formula (I) into an intermediate alkenylboronate of formula (I-M):

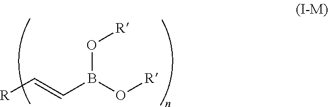

R′OH being chosen in particular from MeOH, EtOH, PrOH or iPrOH;

b) a step of bromination of the intermediate alkenylboronate of formula (I-M) in the presence of CuBr₂, to mainly obtain the (E) bromoalkene;

c) optionally a stage of purification of the bromoalkene (E) obtained, preferably on silica gel; to obtain mainly the (E) isomer; or alternatively:

a) a step of adding dibromine Br₂ to an alkenylaminoborane of formula (I) to form an anti-addition of Br₂ to the alkene function of the alkenylaminoborane and lead to a dibromoalkane via a bridged bromonium,

b) a step of anti-elimination of the aminoborane function and of a bromide of said dibromoalkane, preferably by adding a strong alkoxide base (R″O⁻), preferably the sodium methanolate (MeONa), to mainly obtain the (Z) bromoalkene, c) optionally a step of purification of the (Z) bromoalkene obtained, preferably on silica gel to mainly obtain the (Z) isomer.

Preferably steps a), b) and c) are carried out in one-pot for the two sequences.

Within the meaning of the invention, the term “predominantly” means an isomer content (isomer yield) greater than or equal to 50%, preferably greater than or equal to 70%, even more preferably greater than or equal to 90%.

Another object of the present invention relates to a method for the stereoselective preparation of bromoalkenes (Z) or (E) of the following formulas:

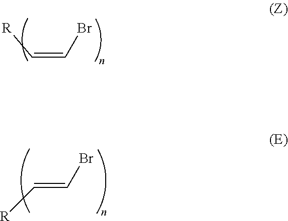

in which R and n have the meanings indicated above, comprising the following steps:

a) a step of preparation of an alkenylaminoborane of formula (I) according to the invention;

b) a step of alcoholysis by an alcohol R′—OH of the alkenylaminoborane of formula (I) into an intermediate alkenylboronate of formula (I-M):

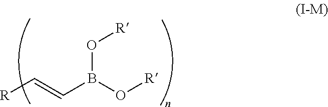

in particular R′OH being chosen from MeOH. EtOH. PrOH or iPrOH;

c) a step of bromination of the intermediate alkenylboronate of formula (I-M) in the presence of CuBr₂, to mainly obtain the (E) bromoalkene;

d) optionally a stage of purification of the (E) bromoalkene obtained, preferably on silica gel; to obtain mainly the (E) isomer; or alternatively:

a) a step for the preparation of an alkenylaminoborane of formula (I) according to the invention;

b) a step of adding dibromine Br₂ to the alkenylaminoborane of formula (I) to form an anti-addition of Br₂ to the alkene function of the alkenylaminoborane and lead to a dibromoalkane via a bridged bromonium,

c) a step of eliminating the anti aminoborane function and of a bromide of said dibromoalkane, preferably by adding a strong alkoxide base (R″O⁻), preferably sodium methanolate (MeONa), to mainly obtain the (Z) bromoalkene,

d) optionally a purification step, preferably on silica gel; to obtain mainly the (Z) isomer.

Preferably steps a), b) and c) are carried out in one-pot for the two sequences.

Preferably, for the sequence of steps leading mainly to the (E) isomer, the alcoholysis step leading to the alkenylboronate of formula (I-M) is carried out by adding 3 equivalents of a molecule of methanol per alkenylaminoborane function and by carrying the reaction medium at −78° C. for one hour.

Preferably, for the sequence of steps leading mainly to the (E) isomer, the bromination step by electrophilic trapping of the alkenylboronate of formula (I-M) is carried out by adding CuBr₂ in a THF:H₂O mixture (1:1) and bringing the reaction medium to 70° C. for 16 hours.

Preferably, for the sequence of steps leading mainly to the (Z) isomer, the step of anti-addition of Br₂ to the alkene function, forming the dibromoalkane via a bridged bromonium, is carried out by introducing the dibromine Br₂ in solution in MTBE at 0° C. and maintaining the reaction medium at 0° C. for one hour.

Preferably, for the sequence of steps leading mainly to the (Z) isomer, the anti-elimination step is carried out by adding MeONa at 0° C. and maintaining the reaction medium at 0° C. for 2 hours.

Without being bound by theory, a reaction mechanism according to the following scheme is proposed for the preparation of a bromoalkene (E) from an alkenylaminoborane of formula (I):

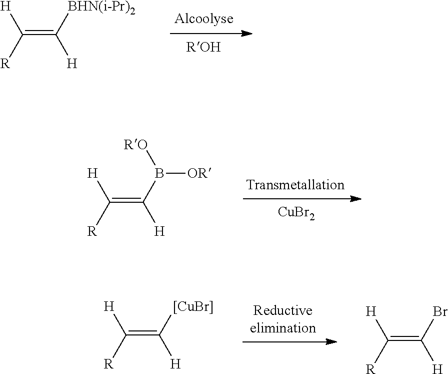

Without being bound by theory, a reaction mechanism according to the following scheme is proposed for the preparation of a bromoalkene (Z) from an alkenylaminoborane of formula (I):

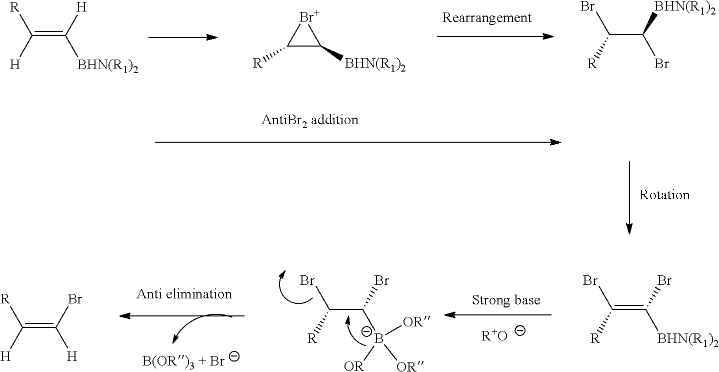

The invention also relates to the use of an alkenylaminoborane of formula (I) prepared according to the method of the invention for the implementation of a stereoselective synthesis of haloalkenes.

The invention also relates to the use of an alkenylaminoborane of formula (I) prepared according to the method of the invention for the implementation of a stereoselective synthesis of alkenes.

The present invention is described below using examples to which it is however not limited.

### DESCRIPTION OF THE PREFERRED EMBODIMENTS

Experimental Part

### Example 1: Preparation of Alkenylaminoboranes of Formula (I): General Protocol for the Preparation of Alkenylaminoboranes

The alkenylaminoboranes of formula (I) are prepared according to the following scheme:

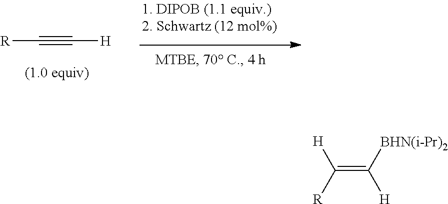

To a solution of terminal alkyne (10 mmol, 1 eq) and of DIPOB (10 mmol, 1.1 eq) in 20 mL of MTBE, is added the Schwartz's reagent in a catalytic quantity (12 mol %).

The mixture is brought to 70° C. for 4 hours.

The products are analyzed by ¹H and ¹¹B NMR.

Conversion Rate:

The conversion is relative to the disappearance of the alkyne.

The conversion rate is determined using the ¹H NMR signals by comparison between the signals of the protons of the alkyne not involved during the reaction which serve as a reference and the signal of the propargyl proton.

Thus a total conversion of 100% corresponds to the total disappearance of the quantity of starting alkyne introduced, indicating that all the alkynes have been transformed during the method.

Tests were carried out with several terminal alkynes having different R groups and the results of the conversion rate of the alkynes are reported in Table 1.

For compound I-I, the degree of conversion (*) is 92% when the reaction is carried out for 4 hours and 100% when the reaction is carried out for 8 hours.

TABLE 1
Alkenylaminoboranes of formula (I)

| Compound | Structure | Conversion (%) |
|---|---|---|
| I-a | 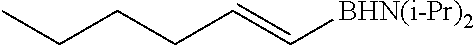 | 100 |
| I-b | 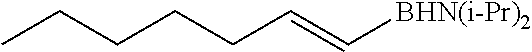 | 100 |
| I-c | 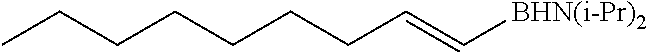 | 100 |
| I-d | 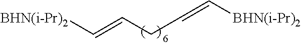 | 100 |
| I-e | 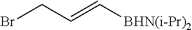 | |
| I-f | 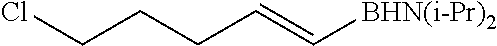 | 100 |
| I-g | 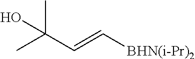 | 100 |
| I-h | 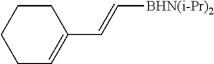 | 100 |
| I-i | 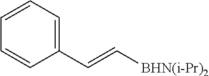 | 100 |
| I-j | 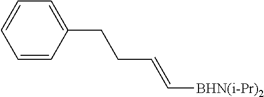 | 100 |
| I-k | 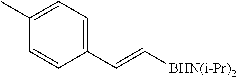 | 100 |
| I-l | 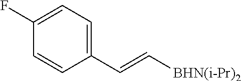 | 92* |
| I-m | 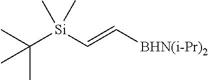 | 100 |
| I-n | 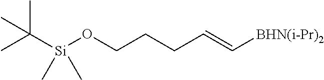 | 100 |
| I-o | 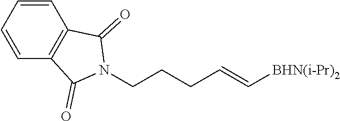 | 100 |
| I-p | 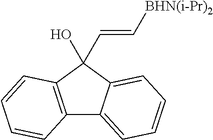 | 100 |
| I-q | 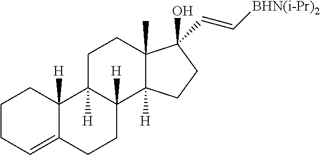 | 100 |

### Example 2: Preparation of Alkenyldiaminoboranes of Formula (II) with Diaminonaphthalene (Dan)

The alkenyldiaminoboranes are prepared according to the following scheme:

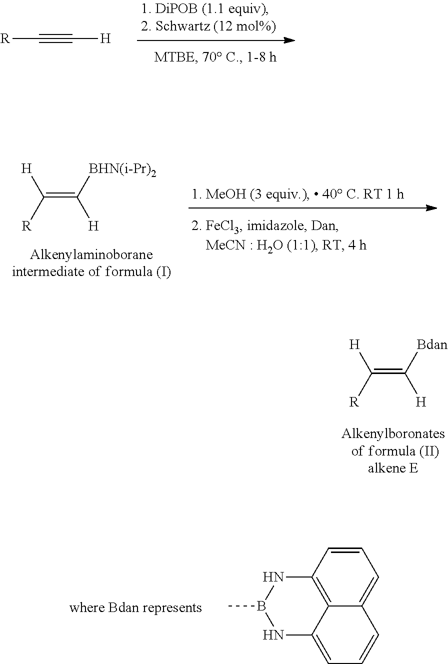

The alkenylaminoborane of formula (I) is prepared according to Example 1 then:

1. a methanolysis (3 equiv.) is carried out at low temperature (−40° C.), for 1 hour, in order to form the intermediate methyl alkenylboronate;

2. the intermediate is subjected to substitution of the methoxyl groups by diaminonaphthalene (dan) in the presence of FeCl₃ and imidazole in a solvent mixture MeCN:H₂O (1:1) at room temperature (RT) for 4 hours.

Tests were carried out with several terminal alkynes having different R groups.

The results of the conversion rate of the alkynes and the yield after purification by column chromatography (silica gel) are reported in Table 2.

TABLE 2
Alkenyldiaminoboranes of formula (II) prepared with diaminonaphthalene (dan)

| Compound | Structure | Conversion (%) | Yield (%) |
|---|---|---|---|
| II-a | 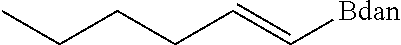 | 100 | 66 |
| II-b | 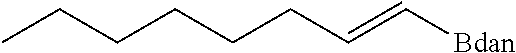 | 100 | 68 |
| II-c | 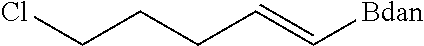 | 100 | 68 |
| II-d | 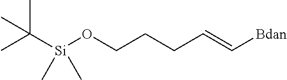 | 100 | 53 |
| II-e | 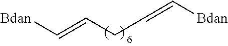 | 100 | 83 |
| II-f | 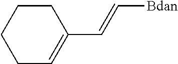 | 100 | 53 |
| II-g | 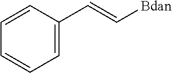 | 100 | 40 |
| II-h | 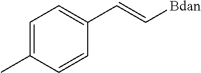 | 100 | 58 |
| II-i | 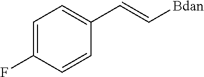 | 100 | 63 |
| II-j | 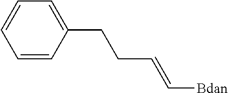 | 100 | 93 |
| II-k | 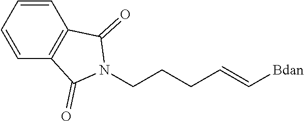 | 100 | 88 |

### Example 3 Preparation of Alkenylboronates of Formula (III) with Pinacol

The alkenylboronates of formula (III) with pinacol are prepared according to the following scheme:

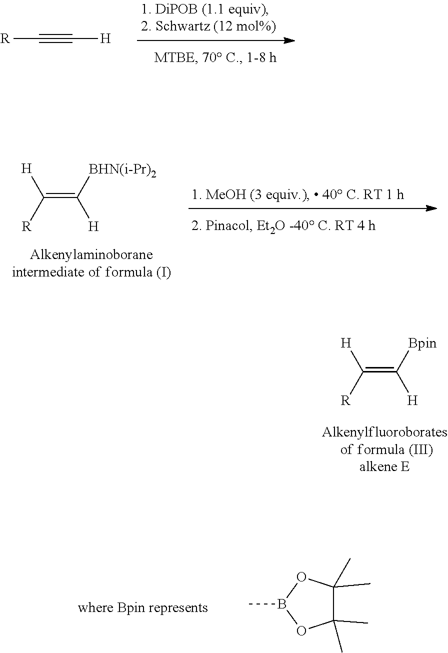

The alkenylaminoborane of formula (I) is prepared according to Example 1 then:

1. a methanolysis (3 equiv.) is carried out at low temperature (−40° C.), for 1 hour, in order to form the intermediate methyl alkenylboronate;

2. the intermediate is transesterified by adding a solution of pinacol in diethyl ether at −40° C., the solution being brought from −40° C. to room temperature (RT), for 4 hours: the methoxyl groups are substituted by pinacol (pin).

Tests were carried out with several terminal alkynes having different R groups.

The results of the degree of conversion of the alkynes and the yield after washing of the products without a purification step are reported in Table 3.

TABLE 3
Alkenylboronates of formula (III) prepared with pinacol

| Compound | Structure | Conversion (%) | Yield (%) |
|---|---|---|---|
| III-a | 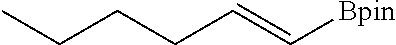 | 100 | 93 |
| III-b | 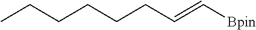 | 100 | 96 |
| III-c | 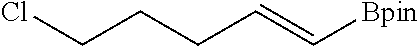 | 100 | 94 |
| III-d | 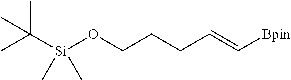 | 100 | 92 |
| III-e | 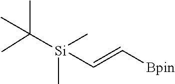 | 100 | 95 |
| III-f | 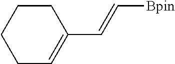 | 100 | 88 |
| III-g | 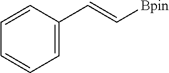 | 100 | 97 |
| III-h | 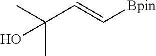 | 100 | 63 |
| III-i | 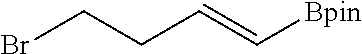 | 100 | 98 |
| III-j | 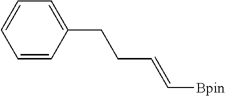 | 100 | 98 |
| III-k | 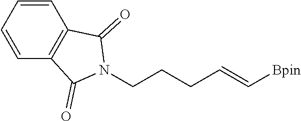 | 100 | 88 |

### Example 4: Preparation of Alkenylboronates of Formula (III) with Neopentylglycol

The alkenylboronates of formula (III) with neopentylglycol are prepared according to the following scheme:

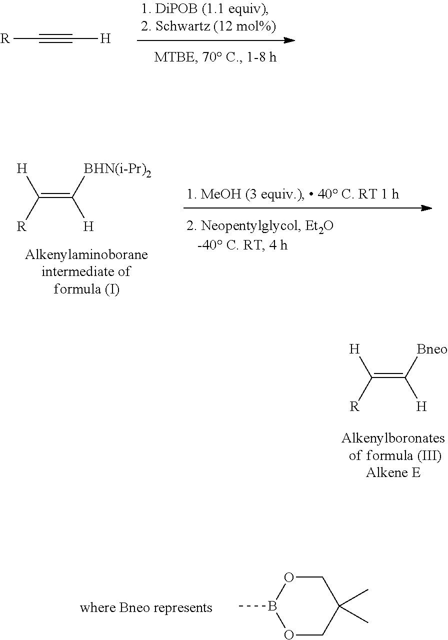

The alkenylaminoborane of formula (I) is prepared according to Example 1 then:

1. a methanolysis (3 equiv.) is carried out at low temperature (−40° C.), for 1 hour, in order to form the intermediate methyl alkenylboronate;

2. the intermediate is transesterified to allow the substitution of the methoxyl groups by neopentylglycol (neo): the neopentylglycol is added in solution in diethyl ether at −40° C. and the solution is brought from −40° C. to room temperature (RT), for 4 hours.

Tests were carried out with several terminal alkynes having different R groups.

The results of the degree of conversion of the alkynes and the yield after washing of the products without a purification step are reported in Table 4.

TABLE 4
Alkenylboronates of formula (III) prepared with neopentylglycol (neo)

| Compound | Structure | Conversion (%) | Yield (%) |
|---|---|---|---|
| III-l | 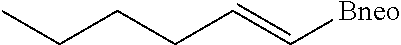 | 100 | 82 |
| III-m | 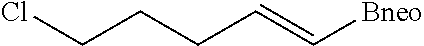 | 100 | 84 |
| III-n | 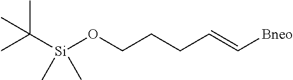 | 100 | 96 |
| III-o | 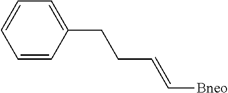 | 100 | 70 |
| III-p | 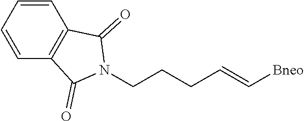 | 100 | 86 |
| III-q | 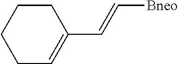 | 100 | 82 |
| III-r Fluorénol | 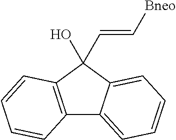 | 100 | 92 |
| III-s Lynestrenol | 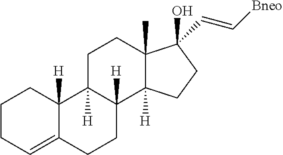 | 100 | 88 |

### Example 5: Preparation of Alkenylfluoroborates of Formula (IV)

The alkenylfluoroborates of formula (IV) are prepared according to the following scheme:

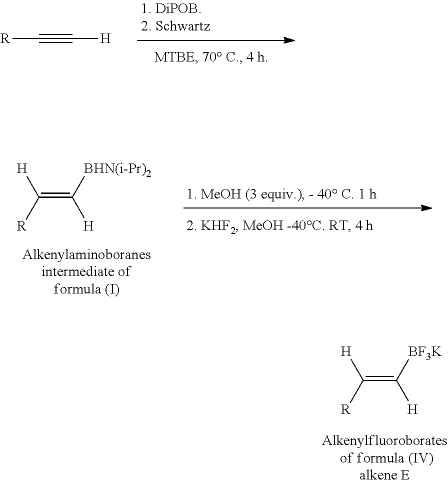

The alkenylaminoborane formula (I) is prepared according to Example 1 then:

1.a methanolysis (3 equiv.) is carried out at low temperature (−40° C.), for 1 hour, in order to form the intermediate methyl alkenylboronate;

2. the intermediate methyl alkenylboronate is transformed into the potassium trifluoroborate salt by substitution of the methoxyl groups, in the presence of a solution of KHF₂ prepared in methanol at −40° C.; the solution is brought from −40° C. to ambient temperature (RT), for 4 h.

Tests were carried out with several terminal alkynes having different R groups.

The results of the conversion rate of alkynes and the yield after precipitation in acetone using diethyl ether are reported in Table 5.

TABLE 5
Alkenylfluoroborates of formula (IV)

| Com- | | Conversion | Yield |
|---|---|---|---|
| pound | Structure | (%) | (%) |
| IV-a | 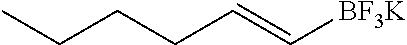 | 100 | 16 |
| IV-b | 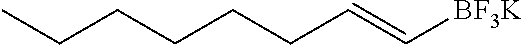 | 100 | 16 |
| IV-c | 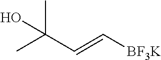 | 100 | 14 |
| IV-d | 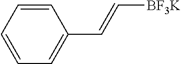 | 100 | 17 |

### Example 6 Synthesis of Alkenylaminoboranes of Formula (I) from DIPOB Generated in Situ

The alkenylboronates of formula (III) are prepared with neopentylglycol from alkenylaminoboranes of formula (I) obtained from DIPOB generated in situ according to the scheme following, steps 1. and 2. being carried out according to the protocol of example 4:

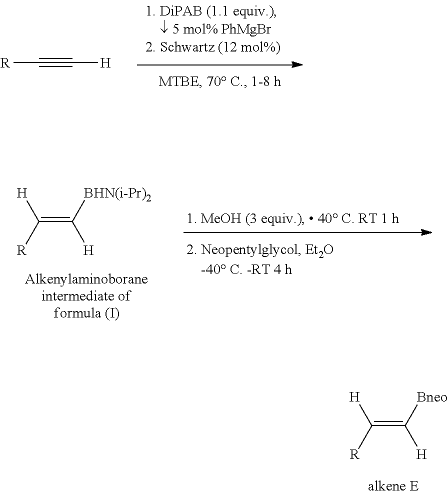

The tests carried out with Fluorenol, Lynestrenol and 4-Phenyl-1-butyne as alkyne, involving the in situ generation of DIPOB via the dehydrogenation of DIPAB in the presence of PhMgBr (5 mol %), allowed quantitative yields, respectively of 92%, 86% and 96%.

### Example 7 Stereoselective Synthesis of Bromoalkenes from the Intermediate of Formula (I)

Preparation of the bromoalkenes (E) The preparation of the bromoalkenes (E) from the intermediate of formula (I) is carried out according to the following scheme:

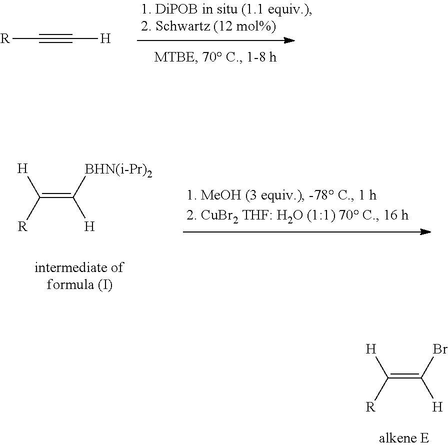

The alkenylaminoborane of formula (I) prepared according to Example 1 undergoes:

1. methanolysis (3 equiv.) at low temperature (−78° C.) for 1 hour to form the intermediate methyl alkenylboronate;

2. Bromination in the presence of CuBr2 in a THF:H20 (1:1) mixture at 70° C. for 16 h.

Tests were carried out with several terminal alkynes having different R groups.

The ratio (Z:E) obtained and the yield after purification on silica gel are reported in Table 6.

TABLE 6
Bromoalkenes (E) obtained from the alkenylaminoborane intermediate of formula (I)

| Compound | Structure | (Z:E) | Yield (%) |
|---|---|---|---|
| V-a | 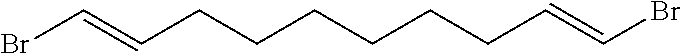 [SMILES: Br/C=C/CCCCCC/C=C/Br] | 2:98 | 56 |
| V-b | 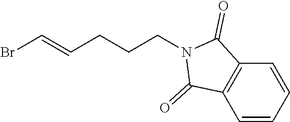 [SMILES: O=C1c2ccccc2C(=O)N1CCC/C=C/Br] | 0:100 | 52 |
| V-c | 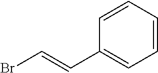 | 0:100 | 72 |
| V-d | 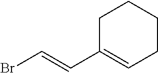 | 0:100 | 62 |
| V-e | 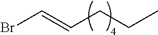 | 0:100 | 39 |
| V-f | 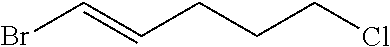 | 6:94 | 61 |
| V-g | 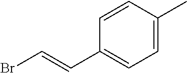 | 0:100 | 45 |
| V-h | 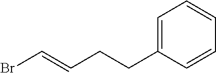 [SMILES: Br/C=C/CCc1ccccc1] | 6:94 | 38 |
| V-i | 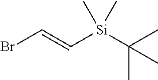 | 5:95 | 41 |

Preparation of Bromoalkenes (Z)

The preparation of the bromoalkenes (Z) from the intermediate of formula (I) is carried out according to the following scheme:

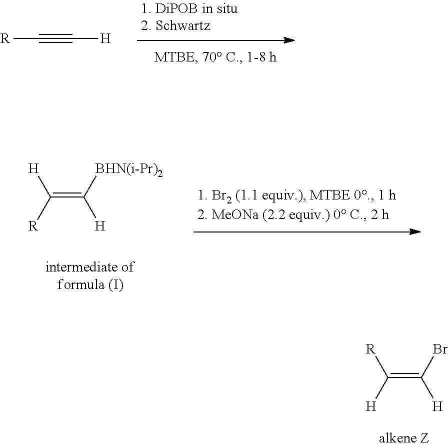

The alkenylaminoborane of formula (c) is prepared according to Example 1 then:

1. Dibromine Br₂ in solution in MTBE at 0° C. is added and the solution is maintained at 0° C. for one hour;

2. MeONa at 0° C. is added and the solution is maintained at 0° C. for 2 h.

Tests were carried out with several terminal alkynes having different R groups.

The ratio (Z:E) obtained and the yield after purification on silica gel are reported in Table 6.

TABLE 7
Bromoalkenes (E) obtained from the alkenylaminoborane intermediate of formula (I)

| Compound | Structure | (Z:E) | Yield (%) |
|---|---|---|---|
| VI-a | 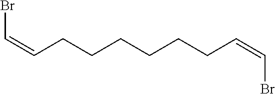 [SMILES: Br/C=C\CCCCCC/C=C\Br] | 96:4 | 84 |
| VI-b | 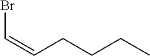 | 100:0 | Non isolé |
| VI-c | 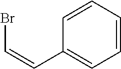 | 70:30 | 75 |
| VI-d | 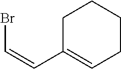 | 53:47 | 88 |
| VI-e | 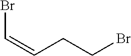 | 94:6 | 66 |
| VI-f | 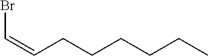 | 100:0 | 78 |
| VI-g | 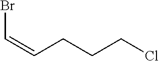 | 99:1 | 78 |
| VI-h | 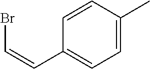 | 50:50 | 79 |
| VI-i | 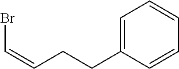 [SMILES: Br/C=C\CCc1ccccc1] | 99:1 | 74 |
| VI-j | 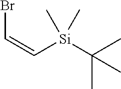 | 99:1 | 46 |

1. Method for the preparation of an alkenylaminoborane of the following formula (I): 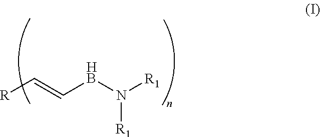
wherein R is:
a linear or branched alkyl group of 1 to 18 carbon atoms optionally bearing at least one substituent,
a linear or branched alkenyl or alkynyl group of 2 to 18 carbon atoms, optionally bearing at least one substituent,
a cycloalkyl or cycloalkenyl group of 3 to 18 carbon atoms, optionally bearing at least one substituent,
a heterocycloalkyl or heterocycloalkenyl group, optionally bearing at least one substituent,
an aryl group of 2 to 12 carbon atoms, where the aryl is chosen from the group of aromatics or heteroaromatics, optionally bearing at least one substituent,
an alkyl aryl group, where the aryl is chosen from the group of aromatics or heteroaromatics, optionally bearing at least one substituent,
a halogen chosen from F, Cl, Br, and I,
a silyl group —SiRₐR_{b}R_{c}, —RₐSiR_{b}R_{c}R_{d}, —RₐOSiR_{b}R_{c}R_{d},
a —ORₐ, —NHRₐ, —NRₐR_{b}, —SRₐ, —CF₃, —NO₂, —RₐOR_{b}, —RₐNHR_{b}, —RₐNR_{b}R_{c}, —RₐSR_{b}, group in which Rₐ, R_{b}, R_{c} and R_{d}, identical or different, represent H, Cl atoms, linear or branched alkyl, alkenyl, alkynyl, cycloalkyl, cycloalkenyl, aryl groups, optionally bearing at least one substituent, where said substituents are chosen from:
a linear, branched or cyclic alkyl groups of 1 to 18 carbon atoms,
the halogens F, Cl, Br and I,
OH,
R₁ is a group chosen from isopropyl, cyclohexyl, n is an integer from 1 to 3,
comprising bringing into contact:
a terminal alkyne, of the following formula: 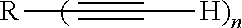
R having the meanings indicated above,
of an aminoborane of formula BH₂—N(R₁)₂,
and a catalyst selected from the group consisting of: Schwartz's reagent (Cp₂ZrHCl), dicyclohexylborane (HBCy), diisopinocamphenylborane (HBipc2), 9-borabicyclo(3.3.1)nonane (9-BBN).

2. Method according to claim 1, wherein the method is carried out at a temperature ranging from 20° C. to 80° C.

3. Method according to claim 1, wherein the catalyst is the Schwartz's reagent of formula (C₅H₅)₂ZrHCl).

4. Method according to claim 1, wherein the catalyst is used in an amount ranging from 0.5% to 20%, in molar percentage.

5. Method according to claim 1, wherein the aminoborane of formula BH₂—N(R₁)₂ is formed in situ by a dehydrogenation reaction of an amine-borane complex of formula H₃B←NH(R₁)₂.

6. Method for the preparation of an alkenylaminoborane of formula (I) according to claim 1, wherein the compound of formula (I) is selected from the group consisting of the following structures:

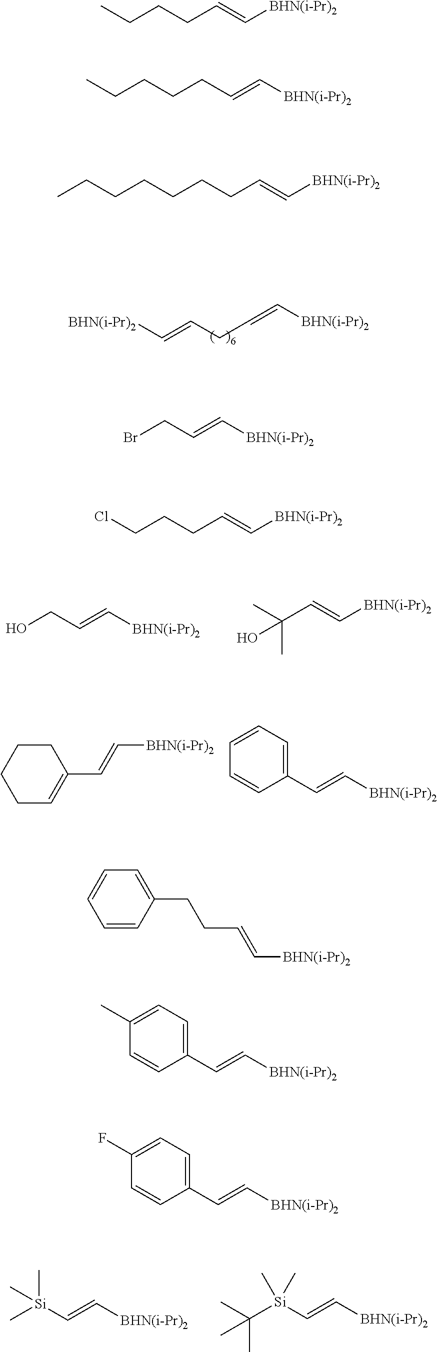

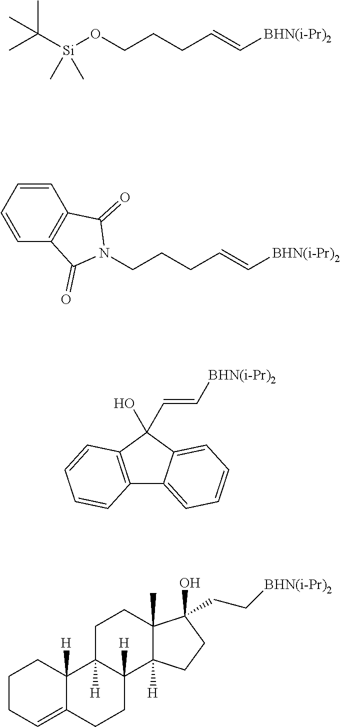

7. Method for the preparation of an alkenylaminoborane of formula (I) according to claim 1, wherein a compound selected from the group consisting of a terminal alkyne, diisopropylaminoborane (DIPOB) or dicyclohexylaminoborane (DICOB) and a catalyst selected from the group consisting of Schwartz's reagent (Cp₂ZrHCl), dicyclohexylborane (HBCy), diisopinocamphenylborane (HBipc2), 9-borabicyclo(3.3.1) nonane (9-BBN), are used for the implementation of said method.

8. Method for the preparation of an alkenylaminoborane of formula (I) according to claim 1 for the preparation of one of the compounds of formulas (II), (III) or (IV) below: 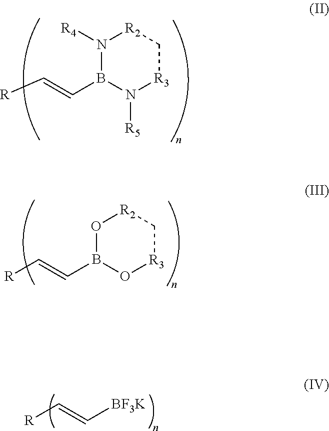
wherein R and n have the meanings given in claim 1,
R₂, R₃, R₄, and R₅, are identical or different and represent hydrogen, linear or branched alkyl, alkenyl, alkynyl, cycloalkyl, cycloalkenyl, aryl groups, or aromatic or non-aromatic heterocyclic groups, with 1 to 18 carbon atoms, optionally bearing at least one substituent, where said substituents are chosen from a linear, branched or cyclic alkyl groups of 1 to 18 carbon atoms, and halogens F, Cl, Br and I, where R₂ and R₃ can be linked to form together a cycle, by implementing a one-pot method.

9. Method for the preparation of an alkenyldiaminoborane compound of formula (II): 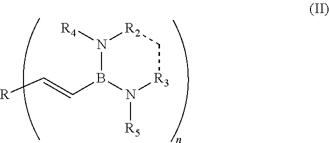
in which
R and n have the meanings given in claim 1,
R₂, R₃, R₄, and R₅ are identical or different and represent hydrogen, linear or branched alkyl, alkenyl, alkynyl, cycloalkyl, cycloalkenyl, aryl groups, or aromatic or non-aromatic heterocyclic groups, with 1 to 18 carbon atoms, optionally bearing at least one substituent, where said substituents are chosen from a linear, branched or cyclic alkyl groups of 1 to 18 carbon atoms, and halogens F, Cl, Br and I, where R₂ and R₃ can be linked to form together a cycle, by implementing a one-pot method, comprising the following steps:
a) a step of preparing an alkenylaminoborane of formula (I) according to claim 1, b) a step of alcoholysis by an alcohol R′—OH of the alkenylaminoborane of formula (I) into an intermediate alkenylboronate of formula (IM): 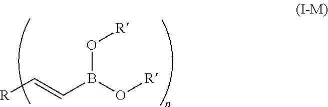
the alcohol R′—OH is chosen from MeOH, EtOH, PrOH or iPrOH,
c) a step of substituting the alkoxyl groups R′O of the compound of formula (IM) with an amino group comprising the groups R₂, R₃, R₄ and R₅ where R₂, R₃, R₄, and R₅ have the meanings indicated above, making it possible to obtain the compound of formula (II),
d) optionally a purification step.

10. Method for the preparation of an alkenyldiaminoborane compound according to claim 9, wherein said amino group is diaminonaphthalene of the following formula:

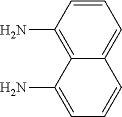 [SMILES: Nc1cccc2cccc(N)c12]

11. Method for the preparation of an alkenylboronate compound of formula (III): 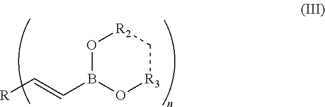
wherein
R and n have the meanings given in claim 1,
R₂ and R₃ are identical or different and represent hydrogen, linear or branched alkyl, alkenyl, alkynyl, cycloalkyl, cycloalkenyl, aryl groups, or aromatic or non-aromatic heterocyclic groups, with 1 to 18 carbon atoms, optionally bearing at least one substituent, where said substituents are chosen from a linear, branched or cyclic alkyl groups of 1 to 18 carbon atoms, and halogens F, Cl, Br and I, where R₂ and R₃ can be linked to form together a cycle, by implementing a one-pot method, comprising the following steps:
a) a step of preparing an alkenylaminoborane of formula (I) according claim 1;
b) a step of alcoholysis by an alcohol R′—OH of the alkenylaminoborane of formula (I) into an intermediate alkenylboronate of formula (I-M): 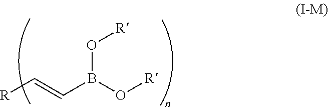
the alcohol is chosen from MeOH, EtOH, PrOH or iPrOH;
c) a step of substituting the alkoxyl groups R′O of the compound of formula (I-M) with an alcohol or diol comprising R₂ and R₃ groups, by a reaction of transesterification;
d) optionally a purification step.

12. Method for the preparation of an alkenylboronate compound of formula (III) according to claim 11, wherein said diol used is pinacol or neopentylglycol.

13. Method for the preparation of an alkenylfluoroborate compound of formula (IV): 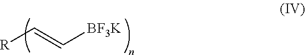
wherein
R and n have the meanings indicated in claim 1, comprising the following steps:
a) a step of preparing an alkenylaminoborane of formula (I) according to claim 1;
b) a step of alcoholysis by an alcohol R′—OH of the alkenylaminoborane of formula (I) into an intermediate alkenylboronate of formula (I-M): 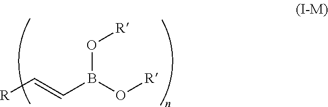
the alcohol R′—OH is chosen from MeOH, EtOH, PrOH or iPrOH;
c) a step of substituting the alkoxyl groups R′O of the compound of formula (I-M) with a fluorinated group;
d) optionally a purification step.

14. Method for the preparation of one of the compounds of formulas (II), (III) or (IV) according to claim 9, corresponding to one of the following structures:

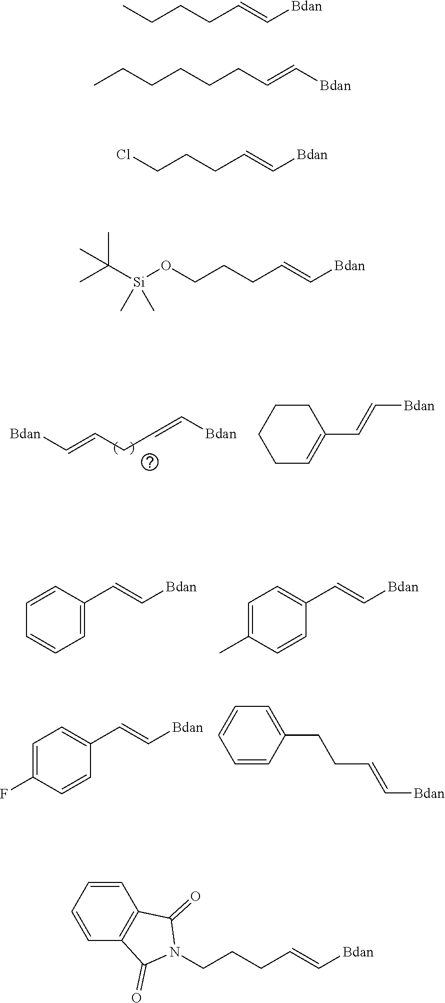

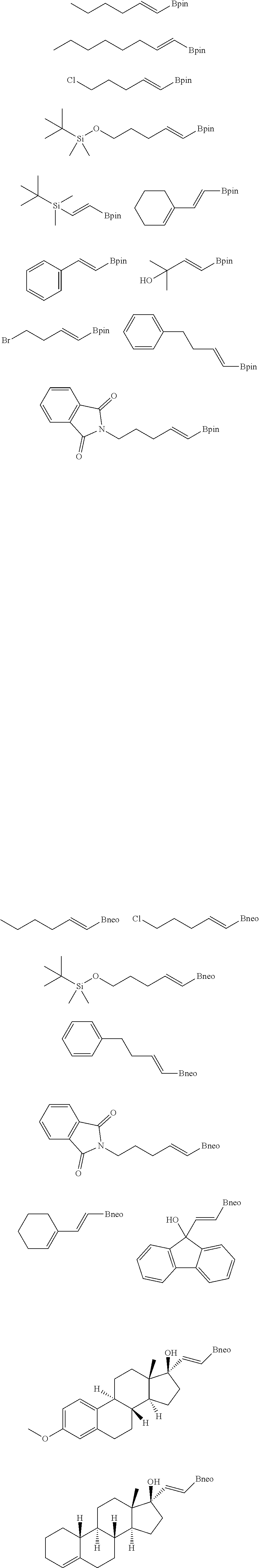

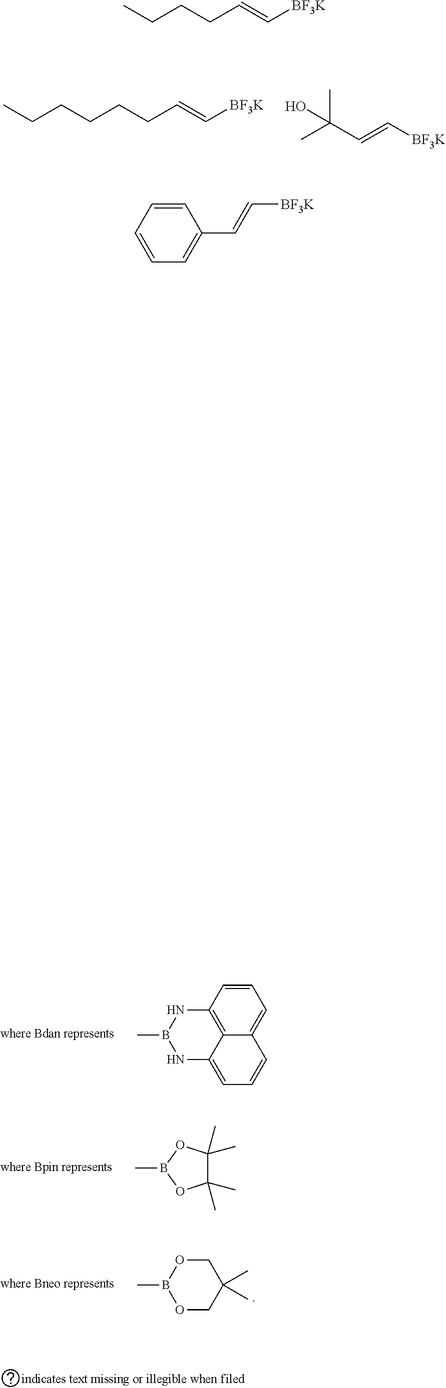

15. Method according to claim 1, wherein the compounds of formulas (I), (II), (III) or (IV), as reaction intermediate compounds, are used for the implementation of stereoselective, multistep or coupling syntheses, selected from the group consisting of Suzuki-Miyaura, Chan-Lam, Petasis and halogenation reactions.

16. Method according to claim 2, wherein the catalyst is the Schwartz's reagent of formula (C₅H₅)₂ZrHCl).

17. Method according to claim 2, wherein the catalyst is used in an amount ranging from 0.5% to 20%, in molar percentage.

18. Method according to claim 3, wherein the catalyst is used in an amount ranging from 0.5% to 20%, in molar percentage.

19. Method according to claim 2, wherein the aminoborane of formula BH₂—N(R₁)₂ is formed in situ by a dehydrogenation reaction of an amine-borane complex of formula H₃B←NH(R₁)₂.

20. Method according to claim 3, wherein the aminoborane of formula BH₂—N(R₁)₂ is formed in situ by a dehydrogenation reaction of an amine-borane complex of formula H₃B←NH(R₁)₂.
